(19) United States
(12) Patent Application Publication
(10) Pub. No.: US 2008/0183057 A1
(43) Pub. Date: Jul. 31, 2008

(54) DISPLAY, DATA STORAGE AND ALARM FEATURES OF AN ADAPTIVE OXYGEN CONTROLLER

(76) Inventor: John TAUBE, Oreland, PA (US)

Correspondence Address: The Law Office of Paul Roath, P.O. Box 427, Norristown, PA, 19404, US

(21) Appl. No.: 11/938,291

(22) Filed: Nov. 11, 2007

Related U.S. Application Data
(60) Provisional application No. 60/858,483, filed on Nov. 13, 2006.

(51) Int. Cl. A61B 5/00

(52) U.S. Cl. 600/323

(57) ABSTRACT

A bar graph display feature for clinical viewing of supplemental oxygen (SpO₂) or blood oxygen percentage, Pulse Rate, and Fraction of Inspired Oxygen (FiO₂) levels as derived from an adaptive supplemental oxygen controller is described. A bar graph is a moving histogram of SpO₂, Pulse Rate, and FiO₂ by using a computer that calculates a FiO₂ by using SpO₂ feedback. The bar graph displays stored data on a flat screen or LCD over specified periods. Other display features include alarm conditions: 1) Upper FiO₂ Limit, 2) Motion Detection, 3) Power Loss, 4) Battery Backup, and 5) Pressure Loss. The Upper FiO₂ Limit is a calculation of FiO₂ by using SpO₂ feedback from a pulse oximeter. The invention also relates to an adaptive oxygen control system whereas adjustment of system time constant and system delay provides application of the control system for use with an oxygen mask, oxyhood or nasal cannula.

### CROSS-REFERENCE TO RELATED APPLICATIONS

This application claims priority to U.S. Provisional Application No. 60/858,483 filed on Nov. 13, 2006, the entire contents of which are incorporated herein by reference.

### BACKGROUND OF THE INVENTION

This invention relates to oxygen control systems for providing supplemental oxygen therapy to patients recovering from respiratory distress and in particular, an adaptive oxygen control system that utilizes SpO₂ feedback from a pulse oximeter to derive the fraction of inspired oxygen delivered to a patient. The display feature for clinical viewing of SpO₂, Pulse Rate, and computer calculated FiO₂ by using SpO₂ from a pulse oximeter is unique and novel in that moving bar histogram of the data is shown to the end user in five minute, one hour, four hour, and eight hour increments. This form of data presentation provides useful information for patient diagnosis and treatment. The data storage feature uses a long-term memory storage device for either data collection and/or data transmission to a hospital information system mainframe by using a USB data port. Data storage of calculated patient parameters such as SpO₂, Pulse Rate, and calculated FiO₂ is a novel means of generating a useful and immediate display of patient parameters. The data can also be used for long-term assessment of patient response to therapy.

An alarm feature provides the end user a novel and useful means to monitor, display and provide corrective actions that relate to potential hazards that effect device operation. These alarm alerts are essential for safe and effective use of an adaptive supplemental oxygen control system.

This invention relates to oxygen control systems for providing supplemental oxygen therapy to patients recovering from respiratory distress and in particular, an adaptive oxygen control system that utilizes SpO₂ feedback from a pulse oximeter to derive the fraction of inspired oxygen delivered to a patient. By adjustment of the system time constant and delay functions, an end user of the oxygen control system can use such a system with a nasal cannula, oxygen mask or oxyhood.

This invention relates to a method of providing diagnostic and/or therapeutic care for long-term oxygen therapy, sleep apnea, oxygen/helium mixture, continuous positive airway pressure, and supplemental oxygen weaning applications.

### DESCRIPTION OF PRIOR ART

An adaptive oxygen control system that utilizes SpO₂ feedback for calculating the fraction of inspired supplemental oxygen delivered to a patient is well known. U.S. Pat. No. 4,889,116 issued to John Taube on Dec. 26, 1989 shows a method and apparatus for the adaptive control of oxygen by using SpO₂ feedback.

U.S. Pat. No. 5,365,922 by Raemer describes a closed loop non-invasive oxygen saturation control system which uses an adaptive controller for delivering a fractional amount of oxygen to a patient. Features of the control algorithm include a method for recognizing when pulse oximeter values deviate significantly from what should be expected. At this point the controller causes a gradual increase in the fractional amount of oxygen delivered to the patient. The feedback control means is also disconnected periodically and the response of the patient to random changes in the amount of oxygen delivered is used to tune the controller response parameters.

U.S. Pat. No. 5,682,877 describes a system and method for automatically selecting an appropriate oxygen dose to maintain a desired blood oxygen saturation level is disclosed. The system and method are particularly suited for use with ambulatory patients having chronic obstructive lung disease or other patients requiring oxygenation or ventilation. In one embodiment, the method includes delivering a first oxygen dose to the patient while repeatedly sequencing through available sequential oxygen doses at predetermined time intervals until the current blood oxygen saturation level of the patent attains the desired blood oxygen saturation levels. The method then continues with delivering the selected oxygen dose to the patient so as to maintain the desired blood oxygen saturation level. U.S. Pat. No. 6,192,883 B1 describes an oxygen control system for supplying a predetermined rate of flow from an oxygen source to a person in need of supplemental oxygen comprising in input manifold, an output manifold and a plurality of gas conduits interconnecting the input manifold to the output manifold. The oxygen source is arranged in flow communication with the input manifold, and a needle valve is positioned in flow control relation to each of the conduits so as to control the flow of oxygen from the input manifold to the output manifold. A plurality of solenoid valves, each having a first fully closed state corresponding to a preselected level of physical activity of the person and a second, fully open state corresponding to another preselected level of physical activity of the person, are positioned in flow control relation to all but one of the conduits. Sensors for monitoring the level of physical activity of the person are provided, along with a control system that is responsive to the monitored level of physical activity, for switching the solenoids between the first state and the second state. A method for supplying supplemental oxygen to a person according to the level of physical activity undertaken by that person is also provided.

World Patent application No. WO 02/056931 A2 by Tyomkin, et al. describes a method for controlling flow of gas to a patient by measuring of a preselected dissolved substance in the blood stream of a patient. The amount of gas is regulated to maintain the preselected dissolved substance above a desired value.

All the patents discussed above are based on controlling a continuous flow of oxygen. There are also patents which have described control algorithms for pulse dose oxygen devices such as the oxygen conserver.

The use of supplemental oxygen to improve oxygen tension and hemoglobin saturation in the blood and decrease the risk of hypoxemia can be associated with oxygen toxicity. In the medical setting mechanical ventilation with 100% inspired oxygen tension can lead to pulmonary toxicity and concomitant pulmonary fibrosis in relatively short periods of time and is a considerable risk in the use of high-dose oxygen in acute medical care. Prolonged breathing of 60-100% oxygen for more than 12 hours will irritate the pulmonary passages, resulting in the Lorraine-Smith effect which is a combination of cough and congestion, sore throat and substemal soreness. After 12 hours, decreased vital capacity occurs which is accompanied by severe pulmonary damage. At greater oxygen tensions, such as hyperbaric oxygen tensions or tensions in which positive end-expiratory pressure ensues, this pulmonary toxicity can be significant and cause sufficient damage in the lungs to offset the benefit of mechanical ventilation with oxygen support. However, oxygen utilization in general aviation for short periods of time, even at 100% oxygen levels, would be expected to have minimal, if any, oxygen toxicity on the subject. Display panels for medical monitoring systems are well known in the art. For example, Cole, et al. has developed a set of objects to display the respiratory physiology of intensive care unit (ICU) patients on ventilators. This set of displays integrates information from the patient, the ventilator, rate of breathing, volume of breathing, and percent oxygen inspired. Using information from object displays, ICU physicians made faster and more accurate interpretations of data than when they used alphanumeric displays. Cole published one study that compared how physicians performed data interpretation using tabular data vs. printed graphical data.

U.S. Pat. No. 6,234,963 describes a system and method for determining and graphically displaying oxygenation states of a patient in real time. The system is non-invasive and can display information to a physician that is intuitive. Various display objects are described for illustrating the output of oxygenation values. The display objects reflect the in vivo physiology that they measure, thus making interpretation of the measured values very intuitive

Electrocardiogram (EKG) monitors are another medical monitoring system that display medical data. EKG data will be printed as a graph on standard paper or shown on the monitor. EKG is the most commonly used diagnostic test in medicine for evaluating the function of the heart. Reading the EKG is very important in patient management, as the difference between a normal and an abnormal reading can be measured in millimeters on the chart.

A variety of electrochemical sensors have been developed for detecting and/or quantifying specific agents or compositions in a patient's blood. Notably, glucose sensors have been developed for use in obtaining an indication of blood glucose levels in a diabetic patient. Such readings are useful in monitoring and/or adjusting a treatment program which typically includes the regular administration of insulin to the patient. Periodic blood glucose readings significantly improve medical therapies using semi-automated medication infusion devices. Some exemplary external infusion devices are described in U.S. Pat. Nos. 4,562,751, 4,678,408 and 4,685,903, while some examples of automated implantable medication infusion devices are described in U.S. Pat. No. 4,573,994, all of which are herein incorporated by reference.

Electrochemical sensors can be used to obtain periodic measurements over an extended period of time. Such sensors can include a plurality of exposed electrodes at one end for subcutaneous placement in contact with a user's interstitial fluid, blood, or the like. A corresponding plurality of conductive contacts can be exposed at another end for convenient external electrical connection with a suitable monitoring device through a wire or cable. Exemplary sensors are described in U.S. Pat. No. 5,299,571, U.S. Pat. Nos. 5,390,671; 5,391,250; 5,482,473; and 5,586,553, which are all incorporated by reference herein.

Devices for measuring various physiological parameters, or “vital signs,” of a patient such as temperature, blood pressure, heart rate, heart activity, etc., have been a standard part of medical care for many years. Indeed, the vital signs of some patients (e.g., those undergoing relatively moderate to high levels of care) typically are measured on a substantially continuous basis to enable physicians, nurses and other health care providers to detect sudden changes in a patient's condition and evaluate a patient's condition over an extended period of time.

The prior art is, however, devoid of a moving histogram display of essential parameters that include SpO₂, Pulse Rate, and calculated FiO₂. These parameters are displayed using five minute, one hour, eight hour, or twenty-four hour increments. A long-term data storage capability is well known. However, use of such data storage of SpO₂, Pulse Rate, and computer calculated FiO₂ is novel, in that for the first time, it is possible for the end user to analyze such data for diagnostic and therapeutic purposes either by visual display and/or utilizing a hospital information sharing system. An alarm display feature that alerts the end user of Upper FiO₂ Limit, Motion Detection, Power Loss, Battery Backup, and Pressure Loss is also well known. What is novel is that such parameters specifically relate to adaptive supplemental oxygen regulation in that each of the described alarms vitally impacts the ability for such oxygen controller to safely and effectively operate as intended.

Similarly, the prior art is devoid of a means to adjust system time constant and delay functions in order to use defined oxygen control system with patients who require a nasal cannula, an oxygen mask or oxyhood for the administration of oxygen therapy.

Finally the prior art does not provide a method of providing diagnostic and/or therapeutic care for long-term oxygen therapy, sleep apnea, oxygen/helium mixture, continuous positive airway pressure, and supplemental oxygen weaning applications.

### OBJECTS OF THE INVENTION

Accordingly, an object of the invention is to provide a new and useful means of a moving histogram displaying critical parameters of computerized supplemental oxygen control system. Displayed parameters include SPO₂, Pulse Rate, and calculated FiO₂ over a five minute, one hour, eight hour, or twenty-four hour increment.

To achieve these and other advantages and in accordance with the purpose of the invention, as embodied and broadly described, one object of the invention is to provide a computer calculated display perimeters by means of a moving histogram over a predetermined time period. The computer calculated display perimeters are FiO₂, SpO₂ and patient pulse rate. The computer calculated display perimeters also provide for a predetermined time period of five minutes, one hour, eight hours or twenty-four hours. The computer calculated display perimeters also provide for alarm and alert conditions detected from a computerized adaptive controller receiving SpO₂ and FiO₂ data. The alarm and alert conditions detected from a computerized adaptive controller receiving SpO₂ and FiO₂ data are Upper FiO₂ Limit, Motion Detection, Power Loss, Battery Backup, Sensor Off Patient, and Pressure Loss.

Another object of the invention is to provide an USB data port access for linking a computerized storage device for the long-term storage of computer calculated display perimeters. The USB data port access for linking a computerized storage device maybe a removable memory device such as a flash drive, a memory card or a memory stick or the storage device may be an external hard drive, a main frame centralized computer or an information management system.

Another object of the invention is to provide a means to adjust system time constant and delay of an adaptive oxygen control system using SPO₂ feedback to use with a nasal cannula, an oxygen mask or oxyhood.

Another object of the invention is to provide a means of long-term memory storage for therapeutic and diagnostic analysis of the patient by means of data review. Data is reviewed either by direct display on the supplemental oxygen delivery system flat screen or LCD, or by a hospital data archiving system.

Another object of the invention is a method for providing Long-Term Oxygen Therapy, HELIOX (oxygen/helium mixture) Therapy, Sleep Apnea Monitoring, Continuous Positive Airway Pressure Therapy, and Weaning from supplemental oxygen.

The accompanying figures are included to provide a further understanding the invention and are incorporated and constitute a part of this specification, illustrate several embodiments of the present invention and together with the description serve to explain the principals of the invention.

### BRIEF DESCRIPTION OF THE DRAWINGS

FIG. 1 is diagram of a touch screen display panel of the computerized adaptive supplementary oxygen control system.

FIG. 2 is diagram of a second touch screen display panel of the computerized adaptive supplementary oxygen control system.

FIG. 3 is diagram of a third touch screen display panel of the computerized adaptive supplementary oxygen control system.

### DETAILED DESCRIPTION

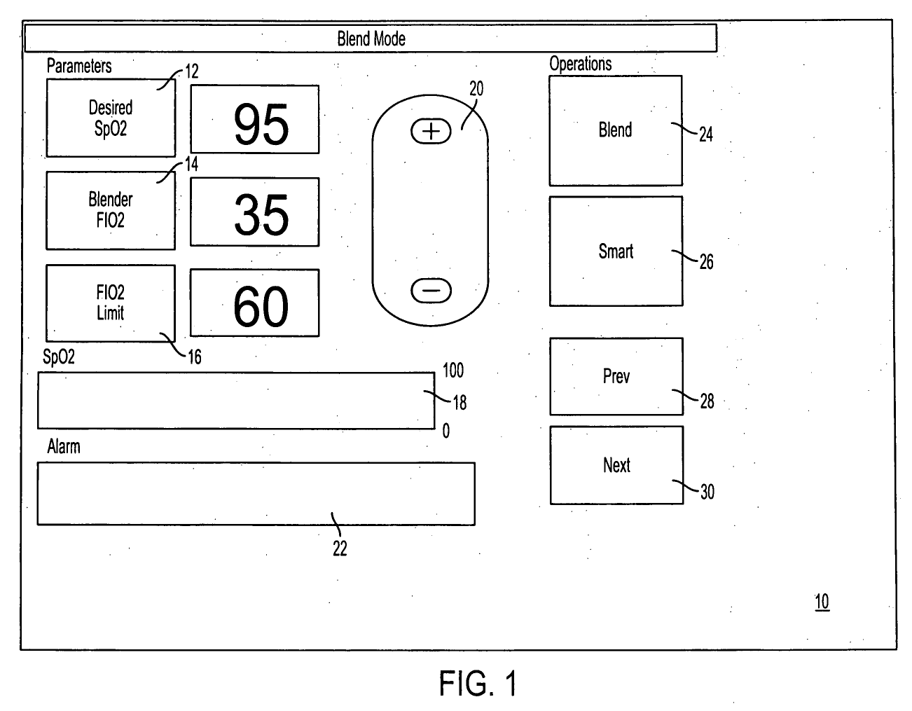

Reference will now be made in detail to the present preferred embodiments of the invention, examples of which are illustrated in the accompanying figures. Referring now in greater detail to FIG. 1, which is a diagram of the initial selection touch screen display panel referred to generally as 10. The operation begins by first selecting the Blend button 24 selection to adjust the desired percentage of SpO₂ by using the desired SpO₂ button 12, ranging from 21% to 100% O₂. The adjustments are made using the adjustment button 20 with the plus (+) or minus (−) buttons. Similarly, adjustments are made to the Blender FiO₂ levels via the Blender FiO₂ button 14 and the FiO₂ limit via the FiO₂ limit button 16. A bar graph 18 appears (shown here the desired SpO₂ level) for each parameter as they are selected and adjusted. Once the adjustments are made, touching the Smart button 26 actives the computer which will automatically monitor the display parameters and adjust the system accordingly. The Prev button 28 and the next button 30 allow the user to toggle between the various screens. Also shown is an alarm button 22 which will turn red if an alarm is triggered. The alarm button, 30, (here depicting low battery) uses a priority means to display operation alarms including Upper FiO₂ Limit, Motion Detection, Power Loss, Battery Backup, and Pressure Loss. If more than one alarm is activated at the same time, an alarm priority is used whereas the alarm with higher priority is displayed

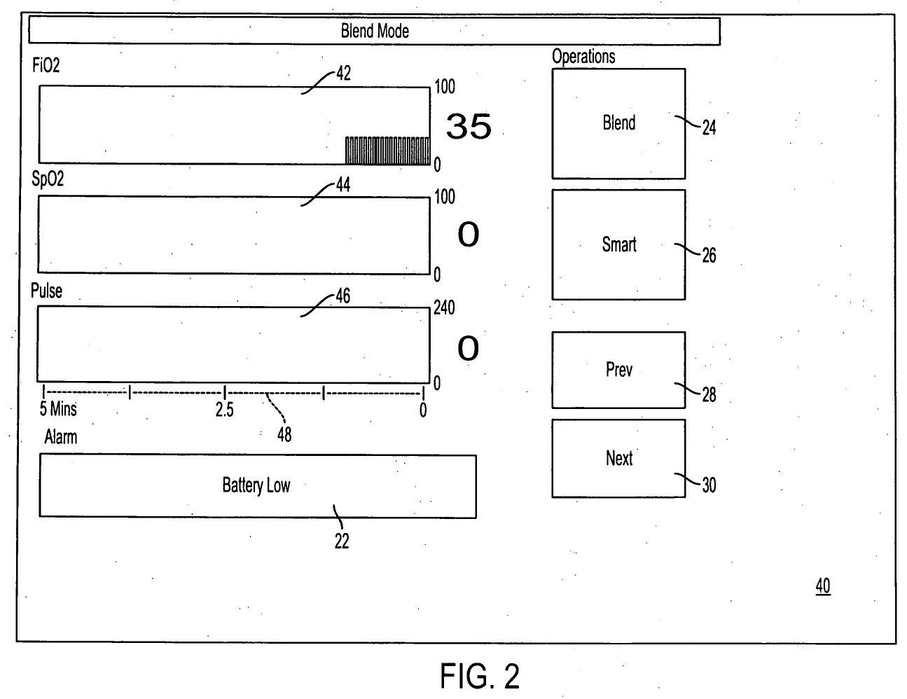

Referring now in greater detail to FIG. 2, generally referred to as 40, the second touch screen display panel is depicted. This touch screen display panel displays the three bar graphs that are histogram displays of the computer calculated FiO₂, SpO₂, and patient pulse rate. The FiO₂ bar 42, the SPO₂ bar graph 44, and the patent's pulse rate 46 are depicted. The time period of the histogram displays can be changed from five minute to one hour, four hour, or eight hour period for diagnostic purposes (here the five minute time period is displayed). Also depicted are the alarm button 22 (here depicting a low battery alarm), Blend button 24, Smart button 26, the Prev button 28 and the next button 30 which all function as previously described above in FIG. 1.

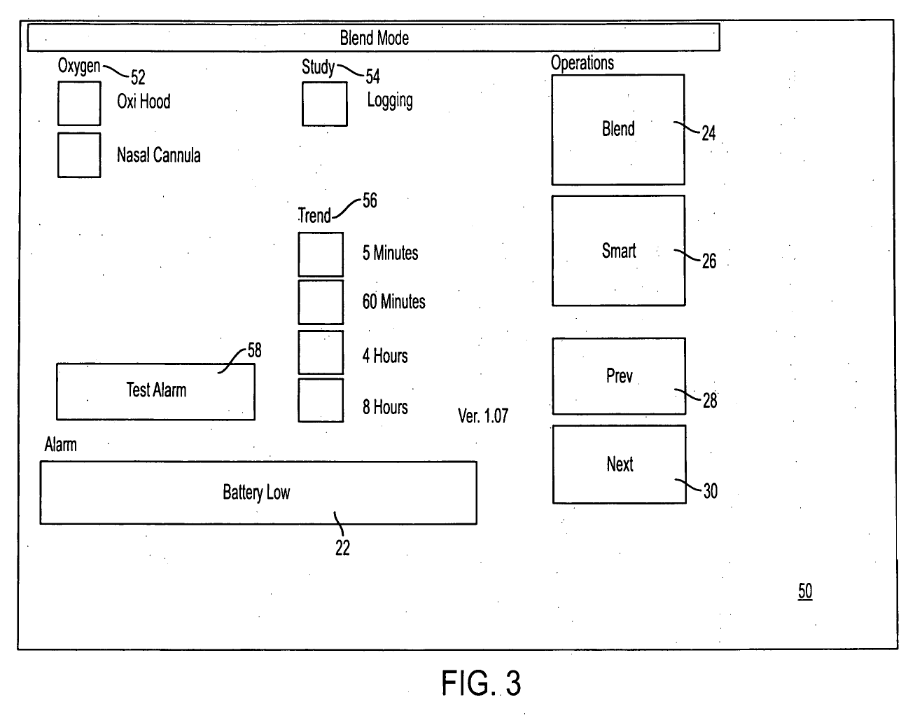

Referring now in greater detail to FIG. 3, generally referred to as 50, the third touch screen display panel is depicted. The Oxygen button 52 allows the user to select between an oxi hood and nasal cannula application depending upon how the supplemental oxygen is delivered to the patent. The Study button 54 activates the computer to track and record the various display parameters (here noted as logging). The Trend button 56 allows the user to select the time period for the bar graphs to display. The trend buttons are five (5) minutes, 60 minutes, four (4) hours and eight (8) hours. The Test Alarm button 58 allows the user to test the alarm to ascertain that the alarm is working. Also depicted are the alarm button 22 (here depicting a low battery alarm), Blend button 24, Smart button 26, the Prev button 28 and the next button 30 which all function as previously described above in FIG. 1.

The moving histogram displays critical parameters of computerized supplemental oxygen control system. Displayed parameters include SpO₂, Pulse Rate, and calculated FiO₂ over a five minute, one hour, eight hour, or twenty-four hour increment. The computer calculated display perimeters also provide for alarm and alert conditions detected from a computerized adaptive controller receiving SpO₂ and FiO₂ data. The alarm and alert conditions detected from a computerized adaptive controller receiving SpO₂ and FiO₂ data are Upper FiO₂ Limit, Motion Detection, Power Loss, Battery Backup, and Pressure Loss.

The present invention provides an USB data port access for linking a computerized storage device for the long-term storage of computer calculated display perimeters. The USB data port access for linking a computerized storage device maybe a removable memory device such as a flash drive, a memory card or a memory stick or the storage device may be an external hard drive, a main frame centralized computer or an information management system.

The present invention also provides a means to adjust system time constant and delay of an adaptive oxygen control system using SpO₂ feedback to use with a nasal cannula, an oxygen mask or oxyhood.

Long-term memory storage for therapeutic and diagnostic analysis of the patient by means of data review is provided by the present invention. Data is reviewed either by direct display on the supplemental oxygen delivery system flat screen or LCD, or by a hospital data archiving system.

1. A computer calculated display perimeters by means of a moving histogram over a predetermined time period.

2. The computer calculated display perimeters according to claim 1, wherein the computer calculated display perimeters are FiO₂, SpO₂ and patient pulse rate.

3. The computer calculated display perimeters according to claim 1, wherein the predetermined time period is selected from group consisting of five minutes, one hour, eight hours or twenty-four hours.

4. The computer calculated display perimeters according to claim 1, wherein the computer calculated display perimeters further comprise alarm and alert conditions detected from a computerized adaptive controller receiving SpO₂ and FiO₂ data.

5. The computer calculated display perimeters according to claim 4, wherein the alarm and alert conditions detected from a computerized adaptive controller receiving SpO₂ and FiO₂ data comprise Upper FiO₂ Limit, Motion Detection, Power Loss, Battery Backup, and Pressure Loss.

6. A USB data port access for linking a computerized storage device for the long-term storage of computer calculated display perimeters.

7. The USB data port access for linking a computerized storage device wherein the computerized storage device comprises a removable memory device.

8. The computerized storage device according to claim 7, wherein the removable memory device comprises a flash drive, a memory card or a memory stick.

9. The computerized storage device according to claim 6, wherein the computerize storage device comprises an external hard drive, a main frame centralized computer or an information management system.

10-18. (canceled)

19. The computerized storage device according to claim 6, wherein the long-term memory storage device data can be reviewed for therapeutic and diagnostic analysis of the patient.

20. The data review according claim 19, wherein the data is reviewed either by direct display on the supplemental oxygen delivery system flat screen or LCD, or by a hospital data archiving system.

21. A means to adjust system time constant and delay of an adaptive oxygen control system using SpO₂ feedback for use with a nasal cannula, an oxygen mask or oxyhood.
